(19) United States
(12) Patent Application Publication
(10) Pub. No.: US 2021/0371714 A1
(43) Pub. Date: Dec. 2, 2021

(54) ADHESIVE FOR ENDOSCOPE, CURED PRODUCT THEREOF, ENDOSCOPE, AND METHOD FOR PRODUCING THE SAME

(71) Applicant: FUJIFILM Corporation, Tokyo (JP)

(72) Inventors: Kazushi FURUKAWA, Ashigarakami-gun (JP); Yoshihiro Nakai, Ashigarakami-gun (JP)

(73) Assignee: FUJIFILM Corporation, Tokyo (JP)

(21) Appl. No.: 17/403,967

(22) Filed: Aug. 17, 2021

Related U.S. Application Data
(63) Continuation of application No. PCT/JP2020/006521, filed on Feb. 19, 2020.

(30) Foreign Application Priority Data
Feb. 26, 2019 (JP) 2019-032976

(51) Int. Cl. C09J 163/00; C08G 59/50; A61B 1/00; C08G 59/06

(52) U.S. Cl. CPC C09J 163/00; C08G 59/06; A61B 1/0011; C08G 59/502

(57) ABSTRACT

An adhesive for an endoscope, the adhesive including an epoxy resin including at least one of bisphenol A epoxy resin, bisphenol F epoxy resin, or phenol novolac epoxy resin, a curing component, and a rubber component; a cured product of the adhesive; an endoscope including the cured product fixed; and a method for producing the endoscope. Note that adhesives in which the rubber component is acrylic rubber are excluded.

### CROSS-REFERENCE TO RELATED APPLICATIONS

This application is a Continuation of PCT International Application No. PCT/JP2020/006521 filed on Feb. 19, 2020, which claims priority under 35 U.S.C. § 119 (a) to Japanese Patent Application No. 2019-032976 filed in Japan on Feb. 26, 2019. Each of the above applications is hereby expressly incorporated by reference, in its entirety, into the present application.

### BACKGROUND OF THE INVENTION

### 1. Field of the Invention

The present invention relates to an adhesive for an endoscope, a cured product of the adhesive, an endoscope, and a method for producing the endoscope.

### 2. Description of the Related Art

Endoscopes for observing, for example, the body cavity, the alimentary canal, or the esophagus of the human body are repeatedly used. For this reason, in such an endoscope, the flexible tube forming the insertion section is, after each use, washed or disinfected using a chemical. In particular, in the cases of insertion into highly susceptible regions such as bronchi, cleanliness of the sterilization grade, which is above the disinfection grade, is required. Thus, endoscopes are required to have high durability for resisting even repeated sterilization treatments using, for example, hydrogen peroxide plasma.

The insertion sections of endoscopes are inserted through, for example, the oral cavity or the nasal cavity, into the body. In order to lessen foreign body sensation and pain of the patient during insertion, the insertion sections of endoscopes desirably have reduced diameters. Thus, members constituting the insertion sections are joined together using, instead of bulky members such as screws and bolts, mainly adhesives.

Among adhesives, epoxy adhesives have high usability, and their cured products are excellent in adhesiveness, electrical characteristics, heat resistance, and moisture resistance, for example. For this reason, epoxy adhesives are used in various fields, and use of epoxy adhesives for fixing constituent members of endoscopes has been studied.

For example, JP2014-210836A states that an adhesive composition contains a base resin including an epoxy resin selected from the group consisting of bisphenol A epoxy resin, bisphenol F epoxy resin, and phenol novolac epoxy resin and acrylic rubber, a curing agent including xylylenediamine, a filler including silica, and an ion exchanger, and states that an adhesive layer formed by curing this adhesive composition, even after a sterilization treatment using hydrogen peroxide plasma, maintains high bonding strength and is less likely to undergo deterioration of the appearance.

### SUMMARY OF THE INVENTION

As described above, for endoscopes, adhesives are frequently used for fixing their constituent members. However, in general, cured products of adhesives tend to have low physical or chemical stability. Thus, in production of devices subjected to high-temperature washing or strong sterilization treatments, such as endoscopes, when members are fixed using adhesives, the devices tend to undergo degradation of the performance after repeated use.

As described above, JP2014-210836A describes a technique of forming, from an epoxy adhesive, an adhesive layer having improved durability for hydrogen peroxide plasma sterilization treatments. In this technique, since deterioration of the adhesive layer during the hydrogen peroxide plasma sterilization treatment is caused by cleavage of polymerized regions of the resin forming the adhesive layer due to the action of the sterilization gas, an ion exchanger is used as a material for trapping the sterilization gas, to thereby achieve the above-described sterilization durability.

Such an adhesive is used for endoscopes not only in fixing of members, but also as a sealing material for filling the gap between an endoscopic member and a support member with a cured product of the adhesive. For example, glass members disposed at the distal end of the insertion section of an endoscope, such as an illumination window and an observation window, are coated, in the peripheries, with the adhesive and fixed at the distal-end portion, and this adhesive also functions as a sealing material to keep airtightness. For example, during washing or disinfection of an endoscope having been used, poor airtightness leads to entry of the liquid into the endoscope, which may cause degradation of the performance and result in failure. The maintaining of airtightness of endoscopes is important from the viewpoint of reliability and safety of endoscopes serving as medical devices.

An object of the present invention is to provide an adhesive for an endoscope, the adhesive being suitable for fixing a constituent member of the endoscope, the adhesive being less likely to deteriorate in a state of being used for fixing of a member (in a state of a cured product) even after repeated sterilization treatments, and a cured product of the adhesive. Another object of the present invention is to provide an endoscope that is, even after repeated sterilization treatments, less likely to undergo degradation of the performance, and a method for producing the endoscope.

In order to achieve the above-described objects, the inventors of the present invention performed thorough studies and, as a result, have found that an epoxy adhesive using a combination of an epoxy resin, a curing component for curing the epoxy resin, and further a rubber component provides a cured product that sufficiently maintains adhesiveness or airtightness even after repeated sterilization treatments using, for example, hydrogen peroxide plasma. On the basis of such findings, they performed further studies and have accomplished the present invention.

The above-described objects have been achieved by the following means.

[1]

An adhesive for an endoscope, the adhesive including an epoxy resin including at least one of bisphenol A epoxy resin, bisphenol F epoxy resin, or phenol novolac epoxy resin, a curing component, and a rubber component,

wherein the rubber component is not acrylic rubber.

[2]

The adhesive for an endoscope according to [1], wherein the rubber component is at least one of natural rubber, diene synthetic rubber, or non-diene synthetic rubber.

[3]

The adhesive for an endoscope according to [1] or [2], wherein the rubber component is at least one of natural rubber, styrene-butadiene rubber, isoprene rubber, butadiene rubber, chloroprene rubber, acrylonitrile-butadiene rubber, butyl rubber, ethylene-propylene rubber, ethylene-propylene-diene rubber, urethane rubber, silicone rubber, fluororubber, chlorosulfonated polyethylene, chlorinated polyethylene, polysulfide rubber, or epichlorohydrin rubber.

[4]

The adhesive for an endoscope according to [1] or [2], wherein the rubber component is non-diene synthetic rubber.

[5]

The adhesive for an endoscope according to any one of [1] to [3], wherein the rubber component is particulate.

[6]

The adhesive for an endoscope according to any one of [1] to [5], wherein the curing component is a polyamine compound.

[7]

The adhesive for an endoscope according to [6], wherein the curing component is a polyether-polyamine compound.

[8]

The adhesive for an endoscope according to any one of [1] to [7], being used as a sealing material.

[9]

A cured product provided by curing the adhesive for an endoscope according to any one of [1] to [8].

An endoscope including a constituent member fixed using the cured product according to [9].

[11]

A method for producing an endoscope, the method including fixing a constituent member using the adhesive for an endoscope according to any one of [1] to [8].

In the descriptions of the present invention, “a value ‘to’ another value” used is intended to include the value and the other value respectively as the lower-limit value and the upper-limit value.

An adhesive for an endoscope according to the present invention is less likely to deteriorate in a state of being used for fixing of a member (in a state of a cured product) even after repeated sterilization treatments. A cured product according to the present invention is less likely to deteriorate even after repeated sterilization treatments. Thus, an endoscope according to the present invention having this cured product as a material for fixing a constituent member is less likely to undergo degradation of the performance even after repeated sterilization treatments. Furthermore, a method for producing an endoscope according to the present invention provides an endoscope that is less likely to undergo degradation of the performance even after repeated sterilization treatments.

### BRIEF DESCRIPTION OF THE DRAWINGS

FIG. 1 is an external view illustrating the configuration of an endoscope according to an embodiment of the present invention;

FIG. 2 is a partial sectional view illustrating the configuration of the insertion section of the endoscope in FIG. 1;

FIG. 3 is an external perspective view illustrating the distal-end portion of the insertion section; and

FIG. 4 is a cutaway partial sectional view illustrating the distal-end portion in which the sections of the lenses and the prism are not hatched.

### DESCRIPTION OF THE PREFERRED EMBODIMENTS

### Adhesive for Endoscope

Preferred embodiments of an adhesive for an endoscope according to the present invention will be described.

An adhesive for an endoscope according to the present invention (hereafter, also referred to as “adhesive according to the present invention”) includes (A) an epoxy resin, (B) a curing component, and (C) a rubber component, wherein the (A) epoxy resin includes at least one of bisphenol A epoxy resin, bisphenol F epoxy resin, or phenol novolac epoxy resin.

The (A) epoxy resin (hereafter, also simply referred to as “Component (A)”) is the base resin of the adhesive. The (B) curing component (hereafter, also simply referred to as “Component (B)”) is a component that reacts with the epoxy resin to cure the adhesive. The (C) rubber component (hereafter, also simply referred to as “Component (C)”) makes the resultant cured product flexible and, as described later, has, for example, a function of relaxing the stress due to deterioration shrinkage to play the role of suppressing deterioration of the cured product.

An adhesive according to the present invention, as long as it includes the above-described components, is not limited in terms of form. For example, an adhesive for an endoscope according to the present invention may have a form containing a mixture of Components (A) to (C) above (one-component type), or may include Components (A) to (C) above such that one or more components among Components (A) to (C) are separated from the other component (two-component type). Alternatively, an adhesive for an endoscope according to the present invention may include Components (A) to (C) such that Components (A) to (C) are separated from each other (three-component type). All these forms fall within the scope of an adhesive according to the present invention.

In this Specification, descriptions of the contents of components in adhesives or definitions of, in the present invention, the contents of components in adhesives are intended that, in the cases of the form of the two-component type or the three-component type,

Components (A) to (C) are mixed at the time of use such that the components satisfy the desired contents in the mixtures. In other words, in the cases of the form of the two-component type or the three-component type, in a state where the components are separated, the contents of Components (A) to (C) do not necessarily satisfy the contents described in this Specification or the contents defined in the present invention. Stated another way, the form of the two-component type or the three-component type satisfies, at the time when Components (A) to (C) are mixed together for use, the contents described in this Specification or the contents defined in the present invention.

When an adhesive for an endoscope according to the present invention is of the one-component type or even the two-component type or the like and includes a mixture of components that react with each other (for example, a mixture of an epoxy resin and a curing component), in order to prevent or sufficiently suppress the reaction between the components to keep a state in which the components are maintained with stability, the adhesive is preferably stored at such a low temperature that the reaction substantially does not occur. For example, the storage can be performed at −20° C. or less, preferably −30° C. or less, more preferably −40° C. or less, still more preferably −50° C. or less. The storage can be performed under light-tight conditions as needed.

An adhesive according to the present invention may include, unless the present invention is hindered from providing advantages, for example, a solvent, a plasticizer, a curing accelerator, an adhesiveness improver (such as a silane coupling agent), a surfactant, a coloring agent (such as a pigment or a dye), an anti-weathering agent, an antioxidant, a heat stabilizer, a lubricant, an antistatic agent, a brightening agent, a release agent, a conductive agent, a viscosity modifier, a filler (such as silica or calcium carbonate), a thixotropy imparting agent, a diluent, and a flame retardant.

An adhesive according to the present invention provides, as a result of a curing reaction, a cured product that is less likely to deteriorate even after repeated sterilization treatments using hydrogen peroxide plasma or the like, to maintain high airtightness, for example. The reason for this is not clarified, but is inferred as follows, for example.

Specifically, degradation of airtightness of the adhesive cured product is caused inferentially as follows: sterilization causes deterioration of the cured product, which causes slight shrinkage of the adhesive cured product; this causes a stress, for example, within the cured product and at the interface between the cured product and a member, which results in generation of gaps. An adhesive according to the present invention includes, in addition to an epoxy resin and a curing component, a rubber component, so that the resultant cured product becomes a cured product having at least partially a flexible region derived from the rubber component; this flexible region relaxes the stress due to deterioration shrinkage, to reduce or suppress the generation of gaps, for example. Note that this reduction, suppression, or the like of the generation of gaps also contributes to maintaining of the adhesiveness of the cured product.

On the other hand, an adhesive containing an epoxy resin, a curing component, and an acrylic rubber component that is not a rubber component according to the present invention provides, as a result of a curing reaction, a cured product that tends to have low airtightness even before sterilization treatments and, after repeated sterilization treatments using, for example, hydrogen peroxide plasma, no longer has sufficient airtightness. The reason for this is not clarified, but is inferred that the acrylic ester structure intramolecularly present in the acrylic rubber component is decomposed by the sterilization treatments and the material has deteriorated.

An adhesive according to the present invention is used to fix various constituent members of an endoscope (endoscope constituent members). Specifically, an adhesive according to the present invention is used to bond an endoscope constituent member to another endoscope constituent member, to thereby fix the endoscope constituent member to the other endoscope constituent member. The adhesive having been used for fixing the endoscope constituent member turns into a cured product to form a bonding portion of the endoscope.

The member fixed using an adhesive according to the present invention is not particularly limited, and preferred examples include metal members, glass members, and resin members. Such an endoscope constituent member is “fixed” by bonding the endoscope constituent member to another constituent member (support member) of the endoscope. Incidentally, the support member may be, for example, the tube wall of the endoscope or an immovable member fixed on the tube wall or the like, or may be a member that is movable to another relative position within the endoscope, such as the tube. In the present invention, the term “fix” is used with meanings including filling, with a cured product of an adhesive, the gap between an endoscope constituent member and a support member into which the member is incorporated, namely, sealing.

Hereinafter, constituent components of an adhesive according to the present invention will be described.

### (A) Epoxy Resin

An adhesive according to the present invention includes, as Component (A), an epoxy resin; this epoxy resin includes at least one of bisphenol A epoxy resin, bisphenol F epoxy resin, or phenol novolac epoxy resin. An adhesive according to the present invention may include one or two or more epoxy resins selected from the group consisting of bisphenol A epoxy resin, bisphenol F epoxy resin, and phenol novolac epoxy resin.

Relative to the total amount of epoxy resin included in an adhesive according to the present invention, the percentage of the total amount of the bisphenol A epoxy resin, the bisphenol F epoxy resin, and the phenol novolac epoxy resin is preferably 70 mass % or more, preferably 80 mass % or more, more preferably 90 mass % or more. More preferably, the epoxy resin included in an adhesive according to the present invention is at least one of bisphenol A epoxy resin, bisphenol F epoxy resin, or phenol novolac epoxy resin.

The epoxy resin included in an adhesive according to the present invention preferably has an epoxy equivalent of 10 to 1000, more preferably 50 to 500, still more preferably 80 to 400, particularly preferably 100 to 300. The epoxy resin included in an adhesive according to the present invention ordinarily has two or more epoxy groups in a single molecule.

The epoxy equivalent is a value obtained by dividing the molecular weight of an epoxy compound by the number of moles of the epoxy groups of the epoxy compound.

The bisphenol A epoxy resin usable for an adhesive according to the present invention is not particularly limited, and such resins commonly used as the base resins of epoxy adhesives can be widely employed. Preferred specific examples include bisphenol A diglycidyl ethers (jER 825, jER 828, and jER 834 (all of which are trade names), manufactured by Mitsubishi Chemical Corporation) and a bisphenol A propoxylate diglycidyl ether (manufactured by Sigma-Aldrich Corporation).

The bisphenol F epoxy resin usable for an adhesive according to the present invention is not particularly limited, and such resins commonly used as the base resins of epoxy adhesives can be widely employed. Preferred specific examples include a bisphenol F diglycidyl ether (trade name: EPICLON 830, manufactured by DIC Corporation) and 4,4′-methylenebis (N,N-diglycidylaniline).

The phenol novolac epoxy resin usable for an adhesive according to the present invention is not particularly limited, and such resins commonly used as the base resins of epoxy adhesives can be widely employed. Such a phenol novolac epoxy resin is, for example, commercially available as product number: 406775 from Sigma-Aldrich Corporation.

The content of the epoxy resin included in an adhesive according to the present invention can be set at 5 to 90 mass %, more preferably 10 to 75 mass %.

### (B) Curing Component

An adhesive according to the present invention contains, as Component (B), one or two or more curing components. The curing component included in an adhesive according to the present invention is not particularly limited, and various curing agents known as curing components for epoxy adhesives can be employed. Examples include acid anhydride compounds, imidazole compounds, phosphorus-containing compounds, polythiol compounds, dicyandiamide compounds, phenolic compounds, and polyamine compounds.

An adhesive according to the present invention, from the viewpoint of greatly maintaining the airtightness even after repeated sterilization treatments, preferably includes, as Component (B), at least one of a polyamine compound, a polythiol compound, or an acid anhydride compound. From the viewpoint of formation of a crosslinking structure more stable against disinfectants used for endoscopes such as peracetic acid, the adhesive more preferably includes, as Component (B), a polyamine compound. Hereinafter, the curing components usable as Component (B) will be described in detail.

(1) Polyamine compound

An adhesive according to the present invention preferably contains, as Component (B), one or two or more polyamine compounds.

Such a polyamine compound is a compound that has, in a single molecule, two or more amino groups having an active hydrogen. This polyamine compound preferably has an unsubstituted amino group (—NH2), more preferably has two or more unsubstituted amino groups. This polyamine compound is still more preferably a primary polyamine compound (a polyamine compound in which all the amino groups are unsubstituted amino groups). For an adhesive according to the present invention, polyamine compounds that exert a curing action in epoxy adhesives can be widely employed.

In a single molecule of the polyamine compound, the number of amino groups having an active hydrogen is preferably 2 to 10, more preferably 2 to 8, still more preferably 2 to 6, still more preferably 2 to 4, particularly preferably 2 or 3. In particular, at least one selected from the group consisting of diamine compounds and triamine compounds can be suitably used.

The polyamine compound preferably has an active-hydrogen equivalent (the equivalent of the active hydrogens of amino groups) of 10 to 2000, more preferably 20 to 1000, still more preferably 30 to 900, still more preferably 40 to 800, still more preferably 60 to 700, particularly preferably 65 to 600.

The active-hydrogen equivalent is a value obtained by dividing the molecular weight of the polyamine compound by the number of moles of active hydrogens of amino groups in the polyamine compound (it means the molecular weight per active hydrogen of amino groups in the polyamine compound).

The polyamine compound preferably has a molecular weight of 100 to 6000, more preferably 100 to 3000. When the polyamine compound is a polymer (for example, in the case of having a polyoxyalkylene group described later), such a molecular weight is a number-average molecular weight.

The polyamine compound preferably has a form in which two or more amino groups are bonded to each other via a group selected from the group consisting of aliphatic hydrocarbon groups, cyclic hydrocarbon groups, aromatic hydrocarbon groups, and heterocyclic groups, or a group of a combination of the foregoing. Such a group may have, in a carbon-carbon bond, a hetero atom such as an oxygen atom, a nitrogen atom, or a sulfur atom (preferably, an oxygen atom).

The polyamine compound, from the viewpoint of being less likely to react with radicals generated by hydrogen peroxide plasma treatments, also preferably does not include, in a carbon-carbon bond, a hetero atom such as an oxygen atom, a nitrogen atom, or a sulfur atom (preferably an oxygen atom). When the polyamine compound does not include a hetero atom in a carbon-carbon bond, the group that bonds together two or more amino groups is more preferably a chain aliphatic hydrocarbon group, and the chain aliphatic hydrocarbon group may have a branch. In such a chain aliphatic hydrocarbon group that may have a branch, the number of carbon atoms is preferably 4 to 50, more preferably 4 to 12, still more preferably 6 to 12.

The polyamine compound, from the viewpoint of imparting further flexibility to the cured product to achieve sturdier properties, also preferably has, in the molecule, a chain alkylene group or an oxyalkylene structure, more preferably has a polyoxyalkylene structure.

The polyamine compound having a chain alkylene group is preferably an alkylenediamine compound. The polyamine compound having a polyoxyalkylene structure (hereafter, also referred to as “polyether-polyamine compound”) is more preferably a polyoxyalkylenediamine compound or a polyoxyalkylenetriamine compound.

The chain alkylene group may be linear or branched, and the number of carbon atoms is preferably 1 to 20, more preferably 5 to 12. Specific examples of the alkylene group include methylene, ethylene, hexamethylene, 2,4,4-trimethylhexamethylene, 2-methylpentamethylene, and dodecamethylene.

The alkylene group of the oxyalkylene structure may be a linear alkylene group or an alkylene group having a branch. In the alkylene group of the oxyalkylene structure, the number of carbon atoms is preferably 1 to 10, more preferably 2 to 6, still more preferably 2 to 4.

The oxyalkylene structure is more preferably an oxyethylene group or an oxypropylene group.

When the polyamine compound of Component (B) has a polyoxyalkylene structure, the plurality of oxyalkylene groups constituting the polyoxyalkylene structure may be the same or different. The average repeating number of the oxyalkylene groups in the polyoxyalkylene structure is preferably 2 to 1000, more preferably 3 to 500. The average repeating number is also preferably 2 to 100, also preferably 2 to 50, also preferably 2 to 35, also preferably 2 to 25. The polyamine compound of Component (B) may have a plurality of polyoxyalkylene structures.

The polyamine compound, from the viewpoint of forming an intramolecular hydrogen bond to provide a material (cured product) having sturdier properties, also preferably has, in the molecule, a polyamide bond (—NH—CO—).

The polyamine compound having a polyamide bond (hereafter, also referred to as “polyamide polyamine compound”) is preferably a polyamide diamine compound.

The average number of amide bonds per molecule of the polyamide polyamine compound is preferably 2 to 50, more preferably 5 to 30, still more preferably 5 to 20.

The linking group that links together the plurality of amide bonds is not particularly limited, and examples include saturated or unsaturated aliphatic hydrocarbon groups and aromatic hydrocarbon groups. When the polyamide polyamine compound has a plurality of linking groups that link together amide bonds, the plurality of linking groups may be the same or different.

The polyamine compound, from the viewpoint of further improving the high airtightness maintained, in a cured product formed from an adhesive according to the present invention, even after repeated sterilization treatments using, for example, hydrogen peroxide plasma, preferably has a polyoxyalkylene structure. When the polyamine compound is a compound having a polyoxyalkylene structure, even after sterilization treatments cause deterioration and decomposition of the crosslinking structure, the cured product has sturdiness and hence is less likely to generate cracking within the cured product, so that the airtightness is inferentially less likely to degrade.

Specific preferred examples of the polyamine compound usable for the present invention are as follows. The numbers at the parentheses are the average repeating numbers of the repeating units within the parentheses.

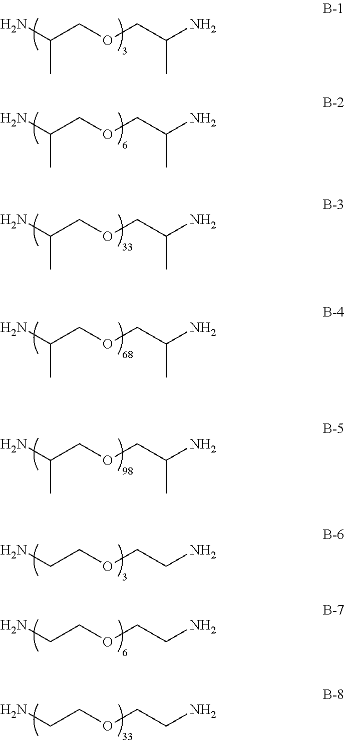

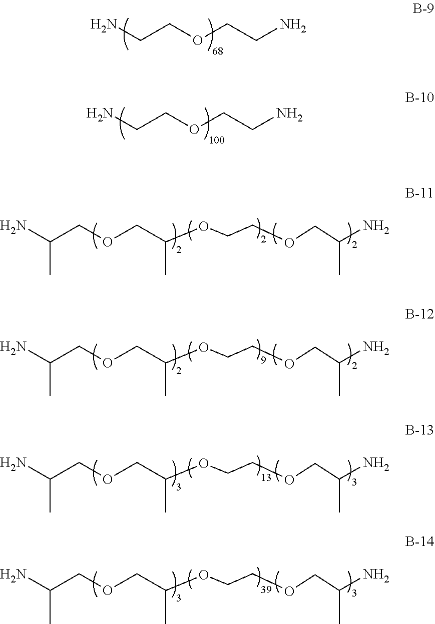

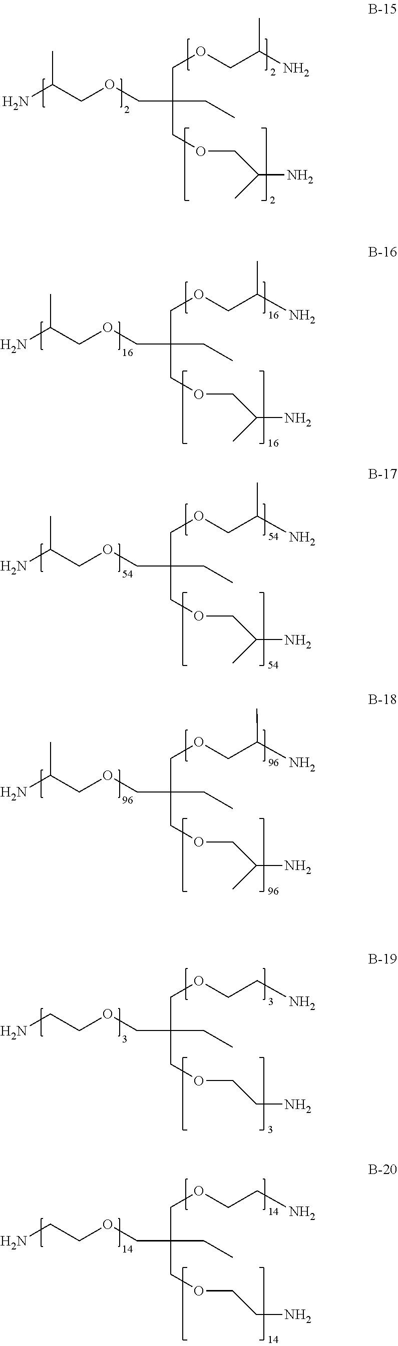

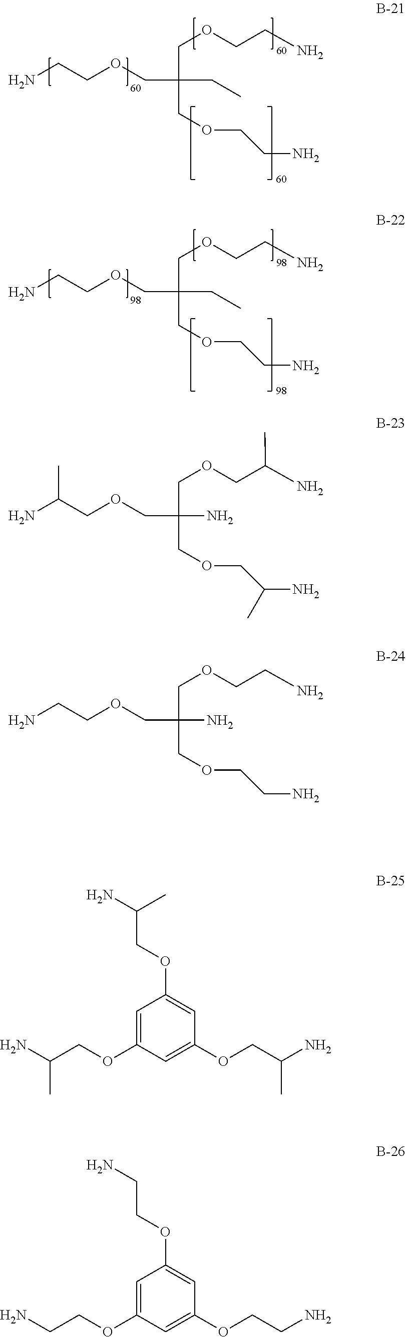

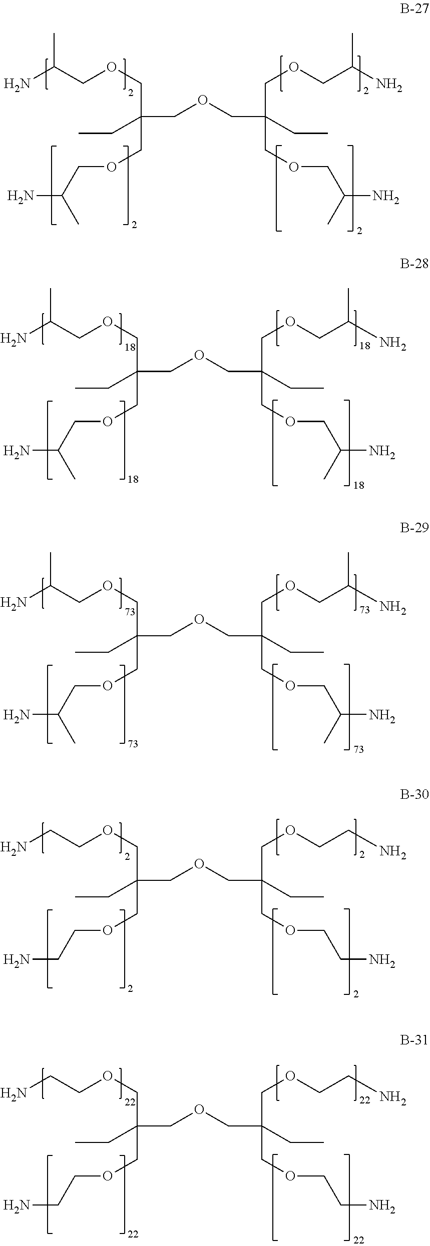

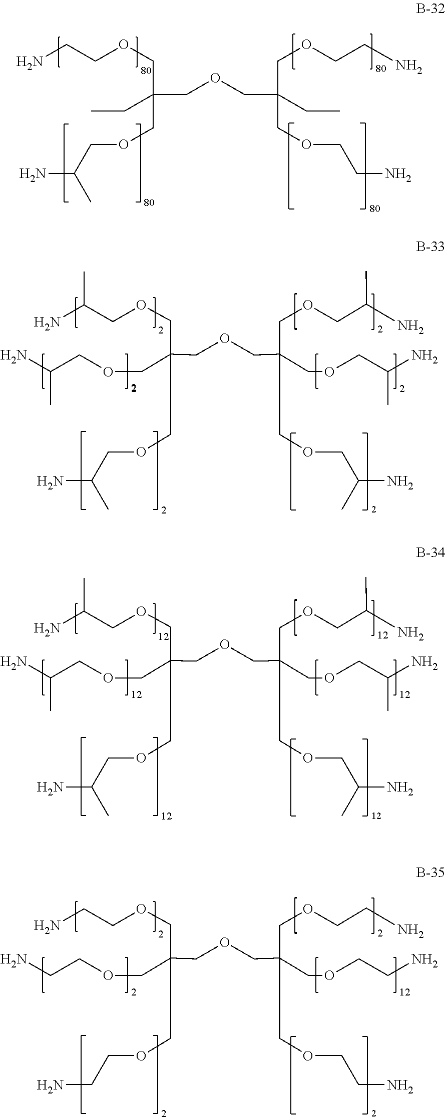

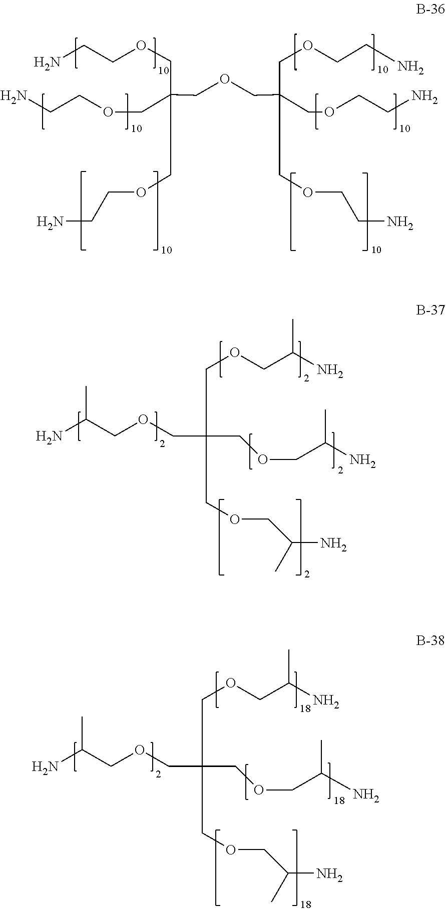

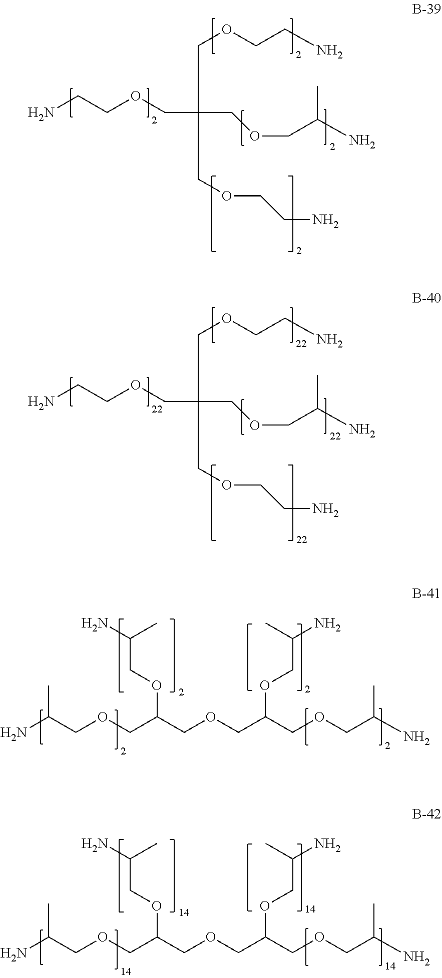

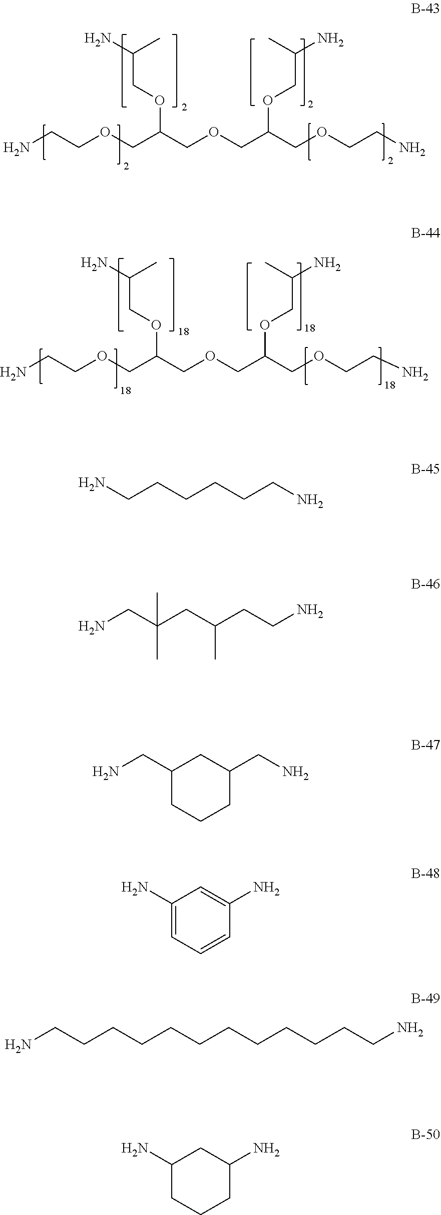

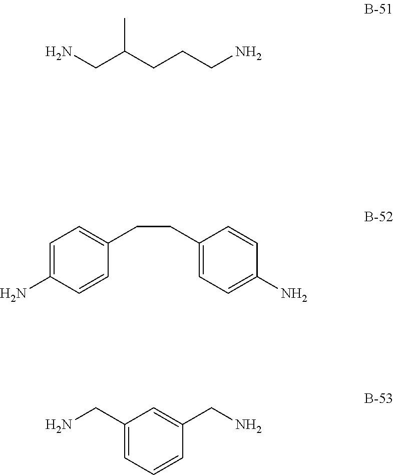

The above-described polyamine compounds can be synthesized in the standard manner. Alternatively, commercially available products may be used.

### (2) Acid Anhydride Compound

An adhesive according to the present invention preferably contains, as Component (B), one or two or more acid anhydride compounds.

Such an acid anhydride compound inferentially causes a copolycondensation reaction with epoxy groups of the epoxy resin serving as Component (A), to cure the epoxy resin. In the present invention, “acid anhydride” means carboxylic anhydride.

Examples of the acid anhydride compound include alicyclic dicarboxylic anhydrides and aromatic cyclic dicarboxylic anhydrides.

Such an alicyclic dicarboxylic anhydride is a compound having a structure in which the carboxy groups at two adjacent carbon atoms constituting the aliphatic ring have undergone dehydration-condensation. Such an aromatic cyclic dicarboxylic anhydride is a compound having a structure in which the carboxy groups at two adjacent carbon atoms constituting the aromatic ring have undergone dehydration-condensation.

The aliphatic ring may or may not partially have, between atoms constituting the ring, a carbon-carbon unsaturated bond, but preferably does not have the carbon-carbon unsaturated bond. This aliphatic ring may be a bridged ring such as a bicyclo ring.

These aliphatic ring and aromatic ring are preferably five-membered rings or six-membered rings.

In particular, these aliphatic ring and aromatic ring are preferably monocyclic, more preferably six-membered rings.

These aliphatic ring and aromatic ring may have one or two or more substituents.

Preferred examples of the substituents that may be present at the aliphatic ring and the aromatic ring include alkyl groups, alkoxy groups, acyl groups, alkoxycarbonyl groups, aryl groups, and carboxy groups. The number of carbon atoms of such an alkyl group is preferably 1 to 10, more preferably 1 to 6, still more preferably 1 to 3, particularly preferably 1 or 2. The number of carbon atoms of such an alkoxy group is preferably 1 to 10, more preferably 1 to 6, still more preferably 1 to 3, particularly preferably 1 or 2. The number of carbon atoms of such an acyl group (including an alkylcarbonyl group and an arylcarbonyl group) is preferably 1 to 30, more preferably 1 to 20, still more preferably 1 to 10. The number of carbon atoms of such an alkoxycarbonyl group is preferably 2 to 30, more preferably 2 to 20, still more preferably 2 to 15, particularly preferably 2 to 4. The number of carbon atoms of such an aryl group is preferably 6 to 20, more preferably 6 to 15, still more preferably 6 to 12, particularly preferably 6.

Among such substituents, two substituents close to each other may be linked to form a ring. Such a ring formed by two substituents linked together is preferably a monocyclic five-membered ring or six-membered ring; two carboxy groups bonded to adjacent atoms constituting the ring are also preferably linked together to form an acid anhydride structure.

A plurality of monovalent or di- or higher valent groups obtained by removing one or two or more hydrogen atoms from such a substituent are also preferably bonded together to form a compound having two or three or more acid anhydride structures in the compound.

The number of the substituents of such an aliphatic ring or aromatic ring is preferably 0 or 1.

The acid anhydride compound is preferably an alicyclic dicarboxylic anhydride.

The number of acid anhydride structures in a molecule of such an acid anhydride compound is preferably 1 to 3, more preferably 1 or 2, still more preferably 1.

Such an acid anhydride compound preferably has a molecular weight of 90 to 800, more preferably 100 to 300.

The acid anhydride compound preferably includes at least one selected from the group consisting of phthalic anhydride, trimellitic anhydride, pyromellitic anhydride, benzophenonetetracarboxylic anhydride, ethylene glycol bisanhydrotrimellitate, glycerol trisanhydrotrimellitate, maleic anhydride, tetrahydrophthalic anhydride, methyltetrahydrophthalic anhydride, methylbutenyltetrahydrophthalic anhydride, hexahydrophthalic anhydride, methylhexahydrophthalic anhydride, succinic anhydride, octenylsuccinic anhydride, dodecenylsuccinic anhydride, methylcyclohexenedicarboxylic anhydride, methylbicyclo[2.2.1]heptane-2,3-dicarboxylic anhydride, and bicyclo[2.2.1]heptane-2,3-dicarboxylic anhydride (hereafter, these acid anhydride compounds will be collectively referred to as Acid anhydride Z). Thus, the acid anhydride compound serving as Component (B) above preferably includes at least one acid anhydride compound selected from Acid anhydride Z.

Acid anhydride Z above is more preferably an acid anhydride compound selected from the group consisting of trimellitic anhydride, hexahydrophthalic anhydride, methylhexahydrophthalic anhydride, succinic anhydride, methylbicyclo[2.2.1]heptane-2,3-dicarboxylic anhydride, bicyclo[2.2.1]heptane-2,3-dicarboxylic anhydride, benzophenonetetracarboxylic anhydride, glycerol trisanhydrotrimellitate, and octenylsuccinic anhydride.

When the acid anhydride compound serving as Component (B) above includes an acid anhydride compound selected from Acid anhydride Z above, this Component (B), unless the present invention is hindered from providing advantages, may include an acid anhydride compound other than Acid anhydride Z. In this case, relative to all the acid anhydride compounds included as Component (B), the percentage of the total amount of the acid anhydride compound selected from Acid anhydride Z is preferably 50 mass % or more, more preferably 70 mass % or more, still more preferably 80 mass % or more, particularly preferably 90 mass % or more. Alternatively, all the acid anhydride compounds included as Component (B) above are also preferably acid anhydride compounds selected from Acid anhydride Z above.

The acid anhydride compound that can be included as Component (B) above, from the viewpoint of transparency of the cured product, preferably does not include nitrogen atoms.

The following are specific examples of acid anhydride compounds usable for the present invention; however, the present invention is not limited to these.

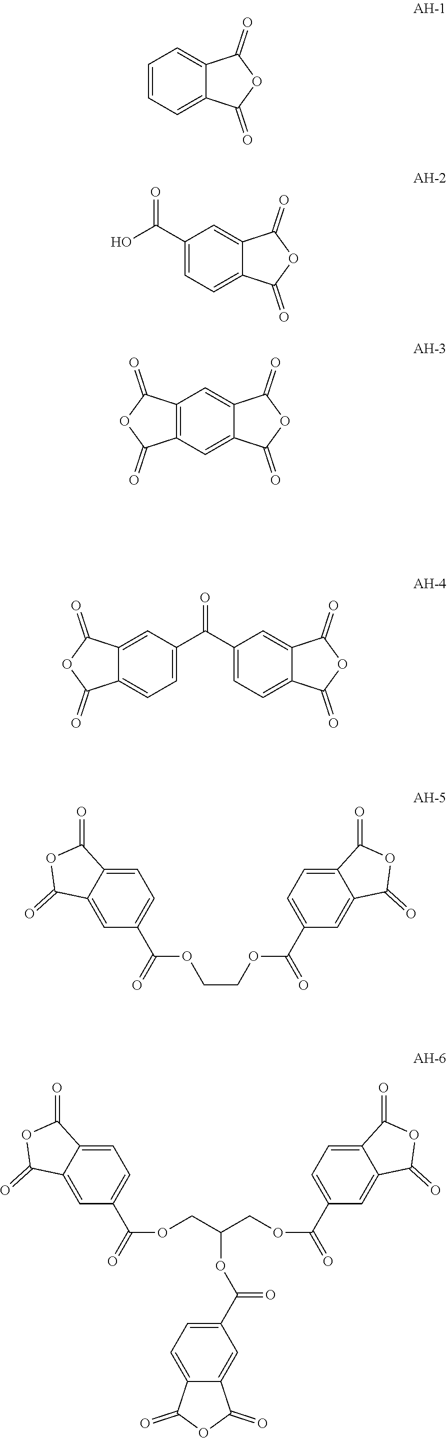

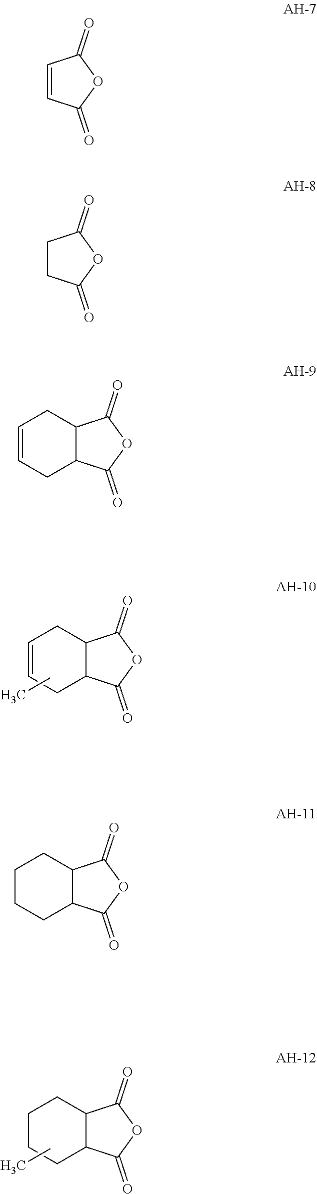

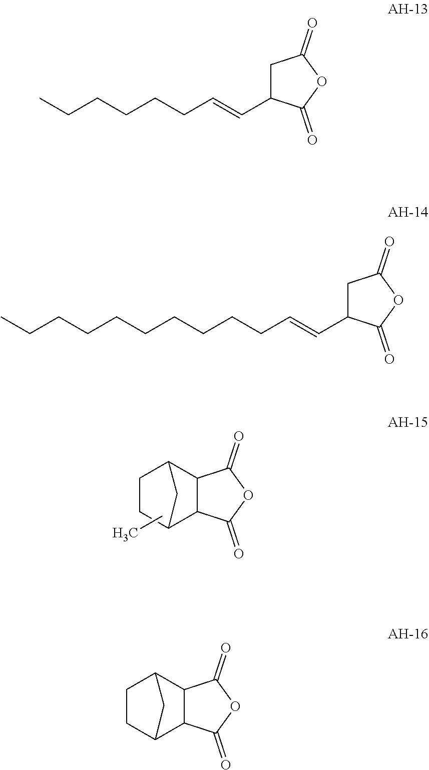

The above-described acid anhydride compounds can be synthesized in the standard manner. Alternatively, commercially available products may be used.

### (3) Thiol Compound

An adhesive according to the present invention preferably contains, as Component (B), one or two or more thiol compounds.

Such a thiol compound is a compound that has at least two moieties represented by General formula (1) below, or at least two moieties represented by General formula (2) below. Such a thiol compound that has a structure having 3 to 10 (preferably 3 to 6) moieties represented by General formula (1) below, or 3 to 10 (preferably 3 to 6) moieties represented by General formula (2) below is preferred from the viewpoint that the cured product has increased crosslinking density to have further improved chemical resistance. On the other hand, such a thiol compound that has a structure having two moieties represented by General formula (1) below, or two moieties represented by General formula (2) below is preferred from the viewpoint that the resultant cured product is relatively flexible to exhibit impact resistance.

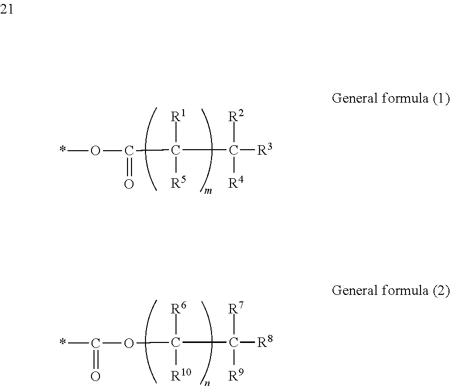

In General formula (1), one of R¹ to R⁵ represents a sulfanyl group (thiol group), and the others each independently represent a hydrogen atom, an alkyl group having 1 to 10 carbon atoms, or an aryl group having 6 to 14 carbon atoms; m represents an integer of 0 to 2. When m is 2, two R¹'s may be the same or different, and two R⁵'s may be the same or different. * denotes the point of attachment in the thiol compound.

In General formula (2), one of R⁶ to R¹⁰ represents a sulfanyl group, and the others each independently represent a hydrogen atom, an alkyl group having 1 to 10 carbon atoms, or an aryl group having 6 to 14 carbon atoms; n represents an integer of 0 to 2. When n is 2, two R⁶'s may be the same or different, and two R¹⁰'s may be the same or different. * denotes the point of attachment in the thiol compound.

The alkyl group having 1 to 10 carbon atoms may be linear or branched, and examples include methyl, ethyl, propyl, i-propyl, butyl, i-butyl, t-butyl, hexyl, and octyl. Of these, methyl or ethyl is preferred.

Specific examples of the aryl group having 6 to 14 carbon atoms include phenyl and naphthyl.

m is preferably 0 or 1.

n is preferably 0 or 1.

The moiety represented by General formula (1) above is preferably a moiety represented by General formula (3) below.

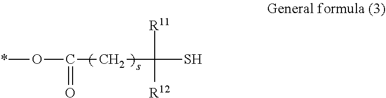

In General formula (3), R¹¹ and R¹² each independently represent a hydrogen atom or an alkyl group having 1 to 10 carbon atoms, and s represents an integer of 0 to 2. * denotes the point of attachment in the thiol compound.

At least one of R¹¹ or R¹² preferably represents an alkyl group having 1 to 10 carbon atoms.

The alkyl groups having 1 to 10 carbon atoms represented by R¹¹ and R¹² have the same definitions and preferred examples as in the above-described alkyl group that can be employed as R¹ in General formula (1).

s is preferably 0 or 1, more preferably 1.

The thiol compound is preferably an ester of a compound represented by General formula (4) below and a polyfunctional alcohol.

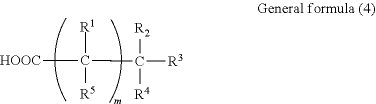

In General formula (4), R¹ to R⁵ and m respectively have the same definitions and preferred examples as in R¹ to R⁵ and m in General formula (1) above.

The compound represented by General formula (4) is preferably a compound represented by General formula (5) below.

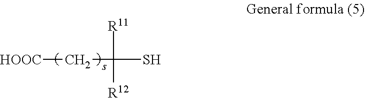

In General formula (5), R¹¹, R¹², and s respectively have the same definitions and preferred examples as in R¹¹, R¹², and s in General formula (3) above.

Specific examples of the compound represented by General formula (4) above include 2-mercaptopropionic acid, 3-mercaptopropionic acid, 3-mercaptobutanoic acid, 2-mercaptoisobutanoic acid, 3-mercapto-3-phenylpropionic acid, 3-mercaptoisobutyric acid, 2-mercapto -3-methylbutyric acid, 3-mercapto-3-methylbutyric acid, 3-mercaptovaleric acid, and 3-mercapto-4-methylvaleric acid.

The polyfunctional alcohol is preferably an alcohol having 2 to 10 functionality (polyol having 2 to 10 hydroxy groups), more preferably 2 to 8 functionality, particularly preferably 2 to 6 functionality.

Specific examples of the polyfunctional alcohol include alkylene glycols (the alkylene groups preferably have 2 to 10 carbon atoms, and the alkylene groups may be linear or branched), diethylene glycol, glycerol, dipropylene glycol, trimethylolpropane, pentaerythritol, and dipentaerythritol.

Examples of the alkylene glycols include ethylene glycol, trimethylene glycol, 1,2-propane glycol, 1,2-butanediol, 1,3-butanediol, 2,3-butanediol, and tetramethylene glycol.

Preferred examples of the polyfunctional alcohol include alkylene glycols having an alkylene main chain having 2 carbon atoms, such as ethylene glycol, 1,2-propane glycol, and 1,2-butanediol, trimethylolpropane, and pentaerythritol.

The following are specific examples of thiol compound usable in the present invention; however, the present invention is not limited to these.

Specific examples include bis(1-mercaptoethyl) phthalate, bis(2-mercaptopropyl) phthalate, bis(3-mercaptobutyl) phthalate, bis(3-mercaptoisobutyl) phthalate, ethylene glycol bis(3-mercaptopropionate), ethylene glycol bis(3-mercaptobutyrate), propylene glycol bis(3-mercaptobutyrate), diethylene glycol bis(3-mercaptobutyrate), tetraethylene glycol bis(3-mercaptopropionate), butanediol bis(3-mercaptobutyrate), octanediol bis(3-mercaptobutyrate), trimethylolpropane tris(3-mercaptobutyrate), trimethylolpropane tris(3-mercaptopropionate), dipentaerythritol hexakis(3-mercaptopropionate), pentaerythritol tetrakis(3-mercaptopropionate), pentaerythritol tetrakis(3-mercaptobutyrate), dipentaerythritol hexakis(3-mercaptobutyrate), ethylene glycol bis(2-mercaptopropionate), propylene glycol bis(2-mercaptopropionate), diethylene glycol bis(2-mercaptopropionate), butanediol bis(2-mercaptopropionate), octanediol bis(2-mercaptopropionate), trimethylolpropane tris(2-mercaptopropionate), pentaerythritol tetrakis(2-mercaptopropionate), dipentaerythritol hexakis(2-mercaptopropionate), ethylene glycol bis(3-mercaptoisobutyrate), propylene glycol bis(3-mercaptoisobutyrate), diethylene glycol bis(3-mercaptoisobutyrate), butanediol bis(3-mercaptoisobutyrate), octanediol bis(3-mercaptoisobutyrate), trimethylolpropane tris(3-mercaptoisobutyrate), pentaerythritol tetrakis(3-mercaptoisobutyrate), dipentaerythritol hexakis(3-mercaptoisobutyrate), ethylene glycol bis(2-mercaptoisobutyrate), propylene glycol bis(2-mercaptoisobutyrate), diethylene glycol bis(2-mercaptoisobutyrate), butanediol bis(2-mercaptoisobutyrate), octanediol bis(2-mercaptoisobutyrate), trimethylolpropane tris(2-mercaptoisobutyrate), pentaerythritol tetrakis(2-mercaptoisobutyrate), dipentaerythritol hexakis(2-mercaptoisobutyrate), ethylene glycol bis(4-mercaptovalerate), propylene glycol bis(4-mercaptoisovalerate), diethylene glycol bis(4-mercaptovalerate), butanediol bis(4-mercaptovalerate), octanediol bis(4-mercaptovalerate), trimethylolpropane tris(4-mercaptovalerate), pentaerythritol tetrakis(4-mercaptovalerate), dipentaerythritol hexakis(4-mercaptovalerate), ethylene glycol bis(3-mercaptovalerate), propylene glycol bis(3-mercaptovalerate), diethylene glycol bis(3-mercaptovalerate), butanediol bis(3-mercaptovalerate), octanediol bis(3-mercaptovalerate), trimethylolpropane tris(3-mercaptovalerate), pentaerythritol tetrakis(3-mercaptovalerate), dipentaerythritol hexakis(3-mercaptovalerate), 1,4-bis(3-mercaptobutyryloxy)butane, 1,3,5-tris(3-mercaptobutyloxyethyl)-1,3,5-triazine-2,4,6(1H,3H,5H)-trione, and tris [(3-mercaptopropionyloxy)ethyl] isocyanurate.

From the viewpoint of having less odor, desired viscosities, and high compatibility with the epoxy resin serving as Component (A), and providing the handleability of a mixture obtained by mixing Component (A) and Component (B), the thiol compound is preferably at least one of 1,4-bis(3-mercaptobutyryloxy)butane, pentaerythritol tetrakis(3-mercaptobutyrate), 1,3,5 -tris(3-mercaptobutyloxyethyl)-1,3,5 -triazine-2 ,4,6(1H,3H,5H)-trione, trimethylolpropane tris(3-mercaptobutyrate), ethylene glycol bis(3-mercaptopropionate), tetraethylene glycol bis(3-mercaptopropionate), trimethylolpropane tris(3-mercaptopropionate), pentaerythritol tetrakis(3-mercaptopropionate), dipentaerythritol hexakis(3-mercaptopropionate), or tris[(3-mercaptopropionyloxy)ethyl] isocyanurate, more preferably at least one of 1,4-bis(3-mercaptobutyryloxy)butane, pentaerythritol tetrakis(3-mercaptobutyrate), 1,3,5 -tris(3-mercaptobutyloxyethyl)-1,3,5 -triazine-2,4,6(1H,3H,5H)-trione, or trimethylolpropane tris(3-mercaptobutyrate).

The molecular weight of the thiol compound is not particularly limited, but is, from the viewpoint of handleability in which, for example, it is easily mixed with the epoxy resin serving as Component (A) of an adhesive according to the present invention and is less likely to separate again, and a mixture obtained by mixing Component (A) and Component (B) is less likely to cause sagging or unevenness, preferably 200 to 1,000, more preferably 300 to 800.

In the present invention, the thiol compound employed may be a commercially available product, and specific examples include 1,4-bis(3-mercaptobutyryloxy)butane (trade name: Karenz MT BD1, manufactured by SHOWA DENKO K. K.), pentaerythritol tetrakis(3-mercaptobutyrate) (trade name: Karenz MT PE1, manufactured by SHOWA DENKO K. K.), 1,3,5-tris(3-mercaptobutyloxyethyl)-1,3,5-triazine-2,4,6(1H,3H,5H)-trione (trade name: Karenz MT NR1, manufactured by SHOWA DENKO K. K.), and trimethylolpropane tris(3-mercaptobutyrate) (trade name: Karenz MT TPMB, manufactured by SHOWA DENKO K. K.).

In Component (B) used in an adhesive according to the present invention, the percentage of the polyamine compound, the acid anhydride compound, and the thiol compound is preferably 80 mass % or more, more preferably 90 mass % or more. Component (B) is also preferably composed only of one or a combination of two or more selected from the group consisting of the polyamine compound, the acid anhydride compound, and the thiol compound. When an adhesive according to the present invention includes, as Component (B), a curing component other than the polyamine compound, the acid anhydride compound, and the thiol compound, as this curing component, the above-described other compounds can be employed.

In an adhesive according to the present invention, the content of Component (B) is not particularly limited, and can be appropriately adjusted in accordance with, for example, the reaction between Component (A) and Component (B).

When Component (B) is the polyamine compound, in an adhesive according to the present invention, the content of the polyamine compound can be appropriately set in consideration of active-hydrogen equivalent, for example.

For example, the content relative to 100 parts by mass of the epoxy resin serving as Component (A) can be set at 5 to 300 parts by mass, more preferably 10 to 250 parts by mass, still more preferably 15 to 220 parts by mass, particularly preferably 15 to 50 parts by mass. The ratio of the active-hydrogen equivalent of the polyamine compound to the epoxy equivalent of the epoxy resin serving as Component (A) (active-hydrogen equivalent/epoxy equivalent) is preferably set at 0.1 to 1.5, more preferably 0.3 to 1.0, still more preferably 0.5 to 1.0.

When Component (B) is the acid anhydride compound, in an adhesive according to the present invention, the content of the acid anhydride compound relative to 100 parts by mass of the epoxy resin serving as Component (A) is preferably 60 to 120 parts by mass, more preferably 70 to 110 parts by mass, still more preferably 80 to 100 parts by mass. When such a mixing ratio is satisfied, the curing reaction and formation of the crosslinking structure are easily controlled, to provide a cured product having high durability.

When Component (B) is the thiol compound, in an adhesive according to the present invention, the content of the thiol compound relative to 100 parts by mass of the epoxy resin serving as Component (A) is preferably 10 to 120 parts by mass, more preferably 15 to 100 parts by mass, still more preferably 20 to 90 parts by mass.

Note that, when Component (B) is the thiol compound, a curing acceleration compound is also preferably used together. The curing acceleration compound is not particularly limited, and examples include amine compounds, guanidine compounds, imidazole compounds, and phosphonium compounds. In this case, in an adhesive according to the present invention, the content of the curing acceleration compound relative to 100 parts by mass of the epoxy resin serving as Component (A) is preferably 0.1 to 10 parts by mass, more preferably 0.2 to 6 parts by mass, still more preferably 0.5 to 3 parts by mass.

### (C) Rubber Component

An adhesive according to the present invention contains, as Component (C), one or two or more rubber components.

The rubber component included in an adhesive according to the present invention is an amorphous and flexible polymer and is, in general, a polymer that has a glass transition temperature of less than room temperature (25° C.) and is in a rubber state at room temperature, preferably a thermosetting polymer, more preferably a polymer that has a tensile elongation of several tens of percent to several hundreds of percent and is highly elastic.

Component (C) above is not particularly limited as long as it is a rubber component that has the above-described properties, and commonly known various rubbers can be employed. However, as Component (C), acrylic rubber is not included. As described above, when adhesives including acrylic rubber are used, after repeated sterilization treatments using, for example, hydrogen peroxide plasma, the airtightness becomes insufficient.

The acrylic rubber means a synthetic rubber that contains acrylic ester as a main component. This phrase “contains acrylic ester as a main component” means containing, relative to all the components of the polymer forming the rubber, 90% or more of acrylic ester components such as alkyl acrylate and aryl acrylate. The term “acrylic ester” includes esters in which at least one of α- or β-carbon atom has a substituent, such as methacrylic ester. Examples of the acrylic rubber normally include copolymer rubbers (ACM) of acrylic ester and 2-chloroethyl vinyl ether, and copolymer rubbers (ANM) of acrylic ester and acrylonitrile.

Component (C) is preferably at least one of (C-0) natural rubber, (C-1) diene synthetic rubber, or (C-2) non-diene synthetic rubber.

In the present invention, (C-0) natural rubber means rubber formed from sap collected from the rubber trees while (C-1) and (C-2) synthetic rubbers mean rubbers synthesized from petroleum or the like.

### (C-1) Diene Synthetic Rubber

The diene synthetic rubber means, of synthetic rubbers, a rubber having a double bond in the polymer main chain forming the rubber.

Specific examples include styrene-butadiene rubber (SBR), isoprene rubber (IR), butadiene rubber (BR), chloroprene rubber (CR), and acrylonitrile-butadiene rubber (NBR).

### (C-2) Non-diene Synthetic Rubber

The non-diene synthetic rubber means, of synthetic rubbers, a rubber that does not have a double bond in the polymer main chain forming the rubber.

Specific examples include butyl rubber (isobutylene-isoprene rubber (IIR)), ethylene-propylene rubber (EPM), ethylene-propylene-diene rubber (EPDM), urethane rubber (U), silicone rubber (also referred to as silicon rubber; (Si, Q)), fluororubber (FKM), chlorosulfonated polyethylene (CSM), chlorinated polyethylene (CM), polysulfide rubber (T), and epichlorohydrin rubber (CO, ECO).

In particular, the non-diene synthetic rubber is preferably used. The non-diene rubber, which substantially does not include, in the polymer chain, a double bond, which tends to be affected by hydrogen peroxide plasma treatments, provide further improved airtightness after sterilization. Of such non-diene rubbers, from the viewpoint of high dispersibility in the epoxy resin serving as Component (A), preferred are butyl rubber (IIR), ethylene-propylene rubber (EPM), and ethylene-propylene-diene rubber (EPDM). From the viewpoint of achieving further improved airtightness after hydrogen peroxide plasma sterilization, more preferred are urethane rubber (U), silicone rubber (Si, Q), and fluororubber (FKM), still more preferred are urethane rubber (U) and silicone rubber (Si, Q).

The rubber component, from the viewpoint of providing further improved airtightness after sterilization treatments, is also preferably particulate. The reason for this is not clarified, but is inferred as follows: when the rubber component is particulate, the rubber component has a small specific surface area, which enables suppression of deterioration of the rubber component itself due to radicals such as hydroxy radicals generated during hydrogen peroxide plasma sterilization treatments, and enables maintaining of the above-described function of relaxing the stress during deterioration shrinkage of the adhesive cured product. Note that, in the case of acrylic rubber particles, the rubber component itself tends to deteriorate, which inferentially results in poor airtightness after repeated sterilization treatments.

The particulate rubber is not particularly limited as long as it is particles including the rubber component. Preferred examples include multilayer-structure fine particles such as core-shell particles having at least a core portion formed of the rubber component and a shell portion formed of a component harder than the rubber component.

The average particle size of the particulate rubber is not particularly limited, and is preferably 50 nm to 2000 nm, more preferably 100 nm to 1000 nm, still more preferably 100 nm to 300 nm. Such average particle sizes are volume-average particle sizes.

As the particulate rubber, commercially available products can also be employed, and examples include METABLEN C-223A (trade name, manufactured by Mitsubishi Chemical Corporation, butadiene rubber (BR) core-shell particles), and KANE ACE MX-960 (trade name, manufactured by KANEKA CORPORATION, dispersion of silicone rubber (Si, Q) core-shell particles in liquid bisphenol A epoxy resin).

In an adhesive according to the present invention, the content of the rubber component is not particularly limited, but, for example, the content relative to 100 parts by mass of the epoxy resin is preferably 1 to 50 parts by mass, more preferably 2 to 40 parts by mass, still more preferably 3 to 30 parts by mass. When the formulation amount is set in such a range, degradation of the strength and adhesiveness is suppressed, to achieve further improved airtightness after sterilization treatments. Note that, when the rubber component is a particulate rubber including a constituent component other than the rubber component, the above-described content of the rubber component means the content of the particulate rubber itself.

### Cured Product

A cured product according to the present invention is a cured product generated by curing an adhesive according to the present invention. Specifically, a cured product according to the present invention is used as a member forming a bonding portion in an endoscope. The curing temperature of an adhesive according to the present invention is not particularly limited, and can be appropriately set in accordance with Component (B) contained in an adhesive according to the present invention. The components can be mixed together in the standard manner. This mixing is preferably performed while bubbles are removed, and hence is ordinarily performed under reduced pressure.

Specifically, in the case of containing, as Component (B), a polyamine compound, in an adhesive according to the present invention, the curing reaction efficiently proceeds even in a low-temperature range to provide a cured product according to the present invention. The curing temperature is, for example, preferably 100° C. or less, more preferably 80° C. or less, still more preferably 60° C. or less, or can be 50° C. or less. In order to sufficiently cause the curing reaction, the curing temperature is preferably 0° C. or more, more preferably 10° C. or more. The curing-reaction time can be appropriately set in accordance with the purpose. Ordinarily, the curing reaction is caused for 1.5 to 200 hours, to provide the cured product.

In the case of containing, as Component (B), an acid anhydride compound, for example, heating at 25 to 220° C. for 0.5 to 48 hours provides a cured product according to the present invention. The curing temperature is preferably 200° C. or less, more preferably 180° C. or less. In order to sufficiently cause the curing reaction, the curing temperature is preferably 120° C. or more, more preferably 140° C. or more.

In the case of containing, as Component (B), a thiol compound, for example, heating at −20 to 150° C. for 10 minutes to 72 hours achieves curing to thereby provide a cured product according to the present invention. The curing temperature is preferably 100° C. or less, more preferably 80° C. or less. In order to sufficiently cause the curing reaction, the curing temperature is preferably 0° C. or more, more preferably 10° C. or more.

Note that the curing temperature of an adhesive according to the present invention is preferably set as low as possible from the viewpoint of reducing repeated exposure of the endoscope to high temperatures during the production steps. Thus, in order to lower the curing temperature, an appropriate curing accelerator is also preferably added.

### Endoscope

In an endoscope according to the present invention, a cured product according to the present invention is used to fix a constituent member. This phrase “a cured product according to the present invention is used to fix a constituent member” means that at least one of the constituent members of the endoscope is fixed to a support member using a cured product according to the present invention.

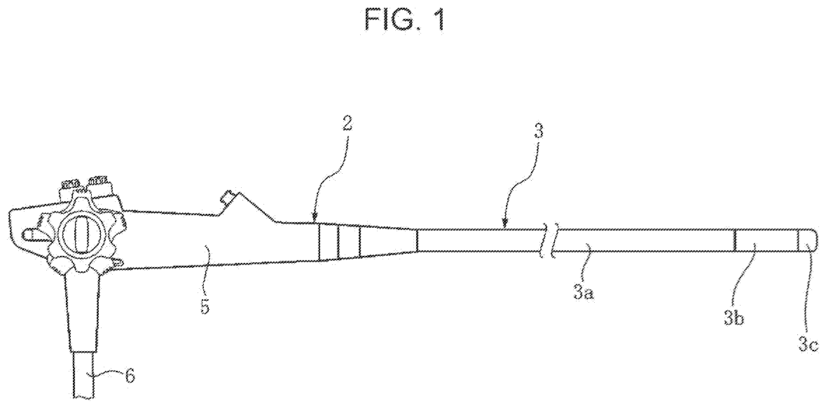

An example of an endoscope (electronic endoscope) according to the present invention will be described. The electronic endoscope includes therein an endoscopic flexible tube (hereafter, the endoscopic flexible tube may be simply referred to as “flexible tube”), and is widely used as a medical device. In the example illustrated in FIG. 1, an electronic endoscope 2 includes an insertion section 3 to be inserted into the body cavity, a main-body operation section 5 coupled to the base-end portion of the insertion section 3, and a universal cord 6 connected to a processor device and a light source device. The insertion section 3 is constituted by a flexible tube 3a coupled to the main-body operation section 5, an angle portion 3b coupled to the flexible tube 3a, and a distal-end portion 3c coupled to the distal end of the angle portion 3b and mainly constituted by metal (such as stainless steel) members. This distal-end portion 3c houses an imaging device (not shown) for imaging the inside of the body cavity. The flexible tube 3a, which accounts for most of the length of the insertion section 3, has flexibility substantially over the whole length; in particular, the portion inserted into inner regions such as the body cavity has a more flexible structure.

In FIG. 1, from the main-body operation section 5 to the distal-end surface of the distal-end portion 3c, a plurality of channels (tubes, not shown) extending throughout in the axial direction of the insertion section 3 are formed.

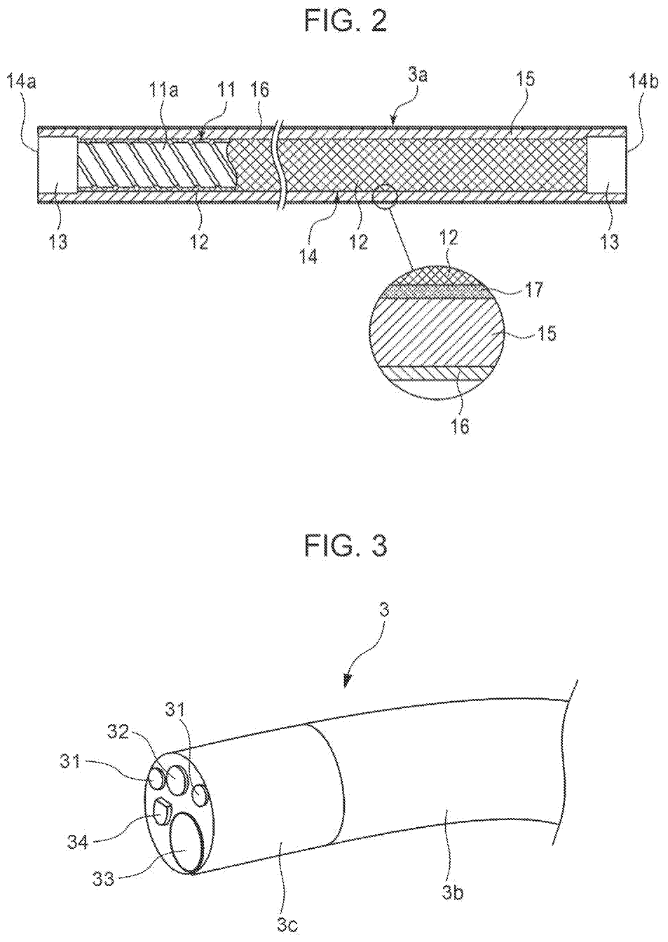

The flexible tube 3a in FIG. 1 has a configuration in which, as illustrated in FIG. 2, the outer peripheral surface of a flexible-tube base 14 is covered with a resin layer 15.

Reference sign 14a denotes the distal-end side (distal-end portion 3c side) while Reference sign 14b denotes the base-end side (main-body operation section 5 side).

The flexible-tube base 14 is formed by covering a spiral tube 11, which is disposed on the innermost side and formed by spirally winding a metal strip 11a, with a sleeve mesh body 12 formed by knitting metal wires. Both ends of the flexible-tube base 14 are fitted with metal caps 13. This resin layer 15 is bonded to the flexible-tube base 14 with an adhesive-cured-product layer 17 therebetween. This adhesive-cured-product layer 17 can be formed by applying and curing an adhesive according to the present invention. The adhesive-cured-product layer (bonding portion) 17 is drawn as a uniform and thick layer for the purpose of illustration, but does not necessarily have this form, and may be disposed in an amorphous form between the resin layer 15 and the flexible-tube base 14. Alternatively, the layer may barely have a thickness and the resin layer 15 and the flexible-tube base 14 may be bonded together substantially in contact with each other.

The outer surface of the resin layer 15 is coated with a coating layer 16 having chemical resistance and containing, for example, fluorine. Incidentally, the adhesive-cured-product layer 17, the resin layer 15, and the coating layer 16 are drawn at large thicknesses relative to the diameter of the flexible-tube base 14 for the purpose of clearly illustrating the layer structure.

As illustrated in FIG. 3, at the distal-end surface of the distal-end portion 3c, illumination windows 31, an observation window 32, and a forceps port 33 are formed. In addition, in order to wash the distal-end surface as needed, a nozzle 34 for sending out water and the air is formed. The illumination windows 31, the observation window 32, the forceps port 33, and the nozzle 34 extend through channels and coupled to the main-body operation section 5.

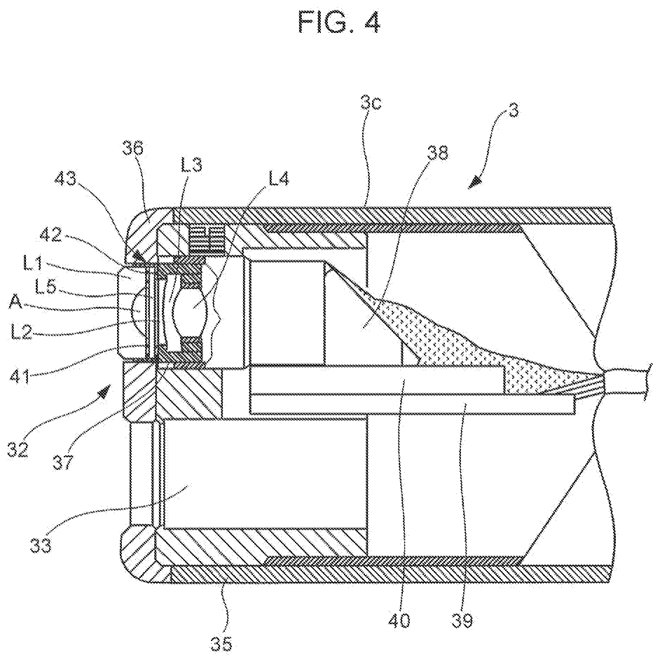

As illustrated in FIG. 4, the distal-end portion 3c is constituted by a distal-end-portion main body 35 formed of metal, and a distal-end cap 36 formed of an electrically insulating member.

At the observation window 32, an observation unit 43, which is an optical device, is disposed. In the observation unit 43, within a lens holder 37, an objective optical system constituted by lenses L1 to L5 is fixed using adhesive cured products 41 and 42. These adhesive cured products 41 and 42 are formed by applying and curing an adhesive according to the present invention. In this objective optical system, Reference sign A denotes an air layer. To an end surface of the lens holder 37, a prism 38 is bonded and fixed. This prism 38 perpendicularly deflects the optical axis of the objective optical system. This prism 38 is fixed to a solid image pickup element 40. The solid image pickup element 40 is fixed to a substrate 39. These can be fixed also with an adhesive according to the present invention.

### Method for Producing Endoscope

A method for producing an endoscope according to the present invention is not particularly limited as long as it includes using an adhesive according to the present invention to fix an endoscope constituent member; as the steps other than the fixing of the endoscope constituent member, ordinary production steps can be employed to produce an endoscope according to the present invention.

The endoscope constituent member to be fixed is not particularly limited in terms of material, and examples include resin members, metal members, and glass members. The endoscope constituent member can be fixed to a support member or the like of an endoscope in the following manner: for example, the components included in an adhesive according to the present invention are mixed together preferably under a reduced pressure; subsequently, the mixture is injected or applied to the application point, and heated, for example, at −10 to 60° C. (preferably 0 to 60° C., more preferably 10 to 50° C.) for 1.5 to 200 hours.

Hereinafter, usage forms of the adhesive in a method for producing an endoscope according to the present invention will be described with reference to specific examples; however, the present invention is not limited to these.

Of endoscope constituent members fixed using an adhesive according to the present invention, a resin member is, for example, a tube inserted through the insertion section of the endoscope. Examples of the resin material forming the tube include fluororesins such as TEFLON (registered trademark), polysulfone, polyesters, polyolefins, and silicone. An adhesive according to the present invention can be used for, for example, bonding between a metal member or a glass member of the insertion section of the endoscope and the tube (fixing of the metal member or the glass member to the tube).

In addition, as described above, the adhesive can also be used to form the adhesive-cured-product layer 17 in FIG. 2. In addition, the adhesive can also be used to bond together, in FIG. 2, the resin layer 15 and the coating layer 16.

An adhesive according to the present invention can be used for outer-surface finishing and fixing of an end portion (distal-end side (angle portion 3b side) end portion of the flexible tube 3a) of the flexible outer cover tube (resin layer 15). Specifically, the end portion of the resin layer 15 of the flexible tube 3a is externally bound tightly using a thread and fixed to the internal member, and subsequently the adhesive is applied so as to cover the thread and cured. The outermost layer of the distal-end-side end portion of the flexible tube 3a is thus formed from an adhesive according to the present invention, so that the thread in this distal-end-side end portion becomes less likely to fray, and the insertion section is easily inserted into the body cavity.

In addition, an adhesive according to the present invention can be used for bonding between the distal-end portion 3c and the angle portion 3b and/or bonding between the insertion section 3 and the main-body operation section 5. For example, the distal-end portion 3c and the angle portion 3b are bonded together using an adhesive according to the present invention; subsequently, the region at and near the bonding portion between the distal-end portion 3c and the angle portion 3b is bound tightly using a thread to reinforce the bonding, and the adhesive is applied so as to cover the thread, and cured. The same applies to bonding between the insertion section 3 and the main-body operation section 5.

In addition, an adhesive according to the present invention can also be used for fixing of various tubes inserted through the insertion section of the endoscope, onto the distal-end portion 3c and/or the main-body operation section 5.

In addition, an adhesive according to the present invention is also preferably used, in the distal-end portion 3c, for sealing of the illumination windows 31 and the observation window 32 (fixing of the glass members). The adhesive can be applied at large thicknesses, to thereby smooth the peripheral corners of the lenses, and to block lateral entry of light into the lenses.

In addition, an adhesive according to the present invention can be used for fixing of members such as assembly of the imaging device housed within the distal-end portion 3c, bonding of parts, and sealing of the solid image pickup element 40. The imaging device has an optical system constituted by a plurality of optical parts such as the lenses L1 to L5 and the prism 38, and the solid image pickup element 40 that photoelectrically converts optical images formed by the optical system into imaging signals, such as a CCD (Charge Coupled Device). An adhesive according to the present invention can be used for, for example, bonding together of optical parts formed of a material such as glass that are the lenses L1 to L5, the prism 38, and the like, and bonding of the lenses L1 to L5, the prism 38, and the like to the substrate 39 formed of resin or metal; this bonding achieves fixing of the glass members, and fixing of the metal members.

In addition, an adhesive according to the present invention can be used for bond-fixing and sealing between the solid image pickup element 40 and the substrate 39. This bonding achieves fixing of the metal members constituting the solid image pickup element, the substrate, and the like.

Thus, a method for producing an endoscope according to the present invention includes a step of using an adhesive according to the present invention to fix an endoscope constituent member.

### EXAMPLES

The present invention will be described further in detail with reference to Examples; however, the present invention is not construed as being limited to Examples below. In Examples below, “room temperature” means 25° C. The formulation amount of each component means the formulation amount of the component itself Specifically, when the raw material includes a solvent, the formulation amount does not include the amount of the solvent.

### PREPARATION EXAMPLE: PREPARATION OF ADHESIVES

The epoxy resins and the rubber components described in Tables below were weighed so as to satisfy the formulation ratios in Tables below, and mixed using a Dissolver (trade name, manufactured by Imoto machinery Co., LTD.) under a condition of 120° C. for 1 hour. The resultant mixtures were further mixed with curing components and, under stirring with an “AWATORI-RENTARO ARV-310 (trade name, manufactured by THINKY CORPORATION)” at room temperature (25° C.), at a reduced pressure of 1.0 Pa, and at a revolution rate of 2000 rpm, defoamed for 5 minutes, to provide adhesives. Note that, in each of the following test examples, such an adhesive immediately after preparation was used.

### TEST EXAMPLES

### Initial Airtightness Test

One of ends of a stainless steel pipe (outer diameter: 4 mm, inner diameter: 2 mm, length: 100 mm) was fitted with a glass lens coated, in the periphery, with an adhesive prepared above; curing at 80° C. for 3 hours was performed to achieve bond-fixing, to produce a lens-equipped pipe. To the other end of the pipe, an air blowing inlet for changing the internal pressure was connected. Subsequently, an airtightness test was performed such that the prepared lens-equipped pipe being submerged in water at 25° C. was left for one minute while an internal pressure of 30 kPa was applied from a side of the pipe opposite from the lens.

Subsequently, the presence or absence of bubbles from the lens-side end was examined. Subsequently, the same test was repeated while the internal pressure was increased in steps of 1 kPa. The minimum internal pressure at which bubbles from the lens-side end were observed was graded in accordance with the following evaluation grades to perform evaluation of initial airtightness. Note that, the higher the internal pressure at which bubbles are starting to be observed, the higher the airtightness. Grades “S” to “B” are pass grades of this test.

### Evaluation Grades

S: Bubbles were not observed even at internal pressures of 45 kPa or more.

A: The internal pressure at which bubbles were starting to be observed was 40 kPa or more and less than 45 kPa.

B: The internal pressure at which bubbles were starting to be observed was 35 kPa or more and less than 40 kPa.

C: Bubbles were observed at internal pressures of less than 35 kPa.

The results are described in Tables below.

### Post-Sterilization-Treatment Airtightness Test

The lens-equipped pipe prepared above was subjected to, using a STERRAD (registered trademark) NX (trade name, manufactured by Johnson & Johnson), a sterilization cycle of the advanced cycle to perform a hydrogen peroxide plasma sterilization treatment. This sterilization treatment was defined as 1 cycle; The lens-equipped pipe after the sterilization treatment was tested as in the initial airtightness test under a condition of an internal pressure of 35 kPa. The maximum number of the cycles at which bubbles were not observed was graded in accordance with the following evaluation grades to perform evaluation of post-sterilization-treatment airtightness. Note that, the larger the number of cycles of the sterilization treatment, the higher the sterilization durability. Grades “S” to “B” are pass grades of this test.

### Evaluation Grades

S: Even after the sterilization treatment was performed for 100 cycles, bubbles were not observed.

A: The number of cycles during which bubbles were not observed was 80 to 99 cycles.

B: The number of cycles during which bubbles were not observed was 50 to 79 cycles. C: The number of cycles during which bubbles were not observed was 1 to 49 cycles.

D: In the pre-sterilization treatment state (initial state), bubbles were observed under a condition of an internal pressure of 35 kPa.

The results are described in Tables below.

TABLE 1-1

| | (A) Epoxy resin | | (B) Curing agent | | (C) Rubber component | | | Evaluation results | |
|---|---|---|---|---|---|---|---|---|---|
| | | Parts by | | Parts by | | Type of | Parts by | Initial | Post-sterilization |
| | Type | mass | Type | mass | Type | rubber | mass | airtightness | airtightness |
| Example 1 | A-2 | 100 | B-1-6 | 40 | C-0 | NR | 15 | B | B |
| Example 2 | A-2 | 100 | B-1-6 | 40 | C-1-1 | SBR | 15 | B | B |
| Example 3 | A-2 | 100 | B-1-6 | 40 | C-1-2 | IR | 15 | B | B |
| Example 4 | A-2 | 100 | B-1-6 | 40 | C-1-3 | BR | 15 | B | B |
| Example 5 | A-1 | 100 | B-1-6 | 40 | C-1-3 | BR | 15 | B | B |
| Example 6 | A-3 | 100 | B-1-6 | 40 | C-1-3 | BR | 15 | B | B |
| Example 7 | A-4 | 100 | B-1-6 | 40 | C-1-3 | BR | 15 | B | B |
| Example 8 | A-5 | 100 | B-1-6 | 40 | C-1-3 | BR | 15 | B | B |
| Example 9 | A-2 | 100 | B-1-6 | 40 | C-1-4 | CR | 15 | B | B |
| Example 10 | A-2 | 100 | B-1-6 | 40 | C-1-5 | NBR | 15 | B | B |
| Example 11 | A-2 | 100 | B-1-6 | 40 | C-2-1 | IIR | 15 | A | A |
| Example 12 | A-2 | 100 | B-1-6 | 40 | C-2-2 | EPM | 15 | A | A |
| Example 13 | A-2 | 100 | B-1-6 | 40 | C-2-3 | EPDM | 15 | A | A |
| Example 14 | A-2 | 100 | B-1-6 | 40 | C-2-4 | U | 15 | S | S |
| Example 15 | A-2 | 100 | B-1-6 | 40 | C-2-5 | Si, Q | 15 | S | S |
| Example 16 | A-2 | 100 | B-1-6 | 40 | C-2-6 | CSM | 15 | A | A |
| Example 17 | A-2 | 100 | B-1-6 | 40 | C-2-7 | CM | 15 | B | A |
| Example 18 | A-2 | 100 | B-1-6 | 40 | C-2-8 | CO, ECO | 15 | B | A |
| Example 19 | A-2 | 100 | B-1-6 | 40 | C-2-9 | FKM | 15 | A | S |
| Example 20 | A-2 | 100 | B-1-6 | 40 | C-3-1 | BR (particles) | 15 | S | S |
| Example 21 | A-2 | 100 | B-1-6 | 40 | C-3-2 | Si, Q (particles) | 15 | S | S |
| Example 22 | A-2 | 100 | B-1-6 | 40 | C-2-1 | IIR | 1 | B | B |
| Example 23 | A-2 | 100 | B-1-6 | 40 | C-2-1 | IIR | 5 | B | A |
| Example 24 | A-2 | 100 | B-1-6 | 40 | C-2-1 | IIR | 50 | B | B |
| Example 25 | A-2 | 100 | B-1-6 | 40 | C-2-1 | IIR | 65 | B | B |

TABLE 1-2

| | (A) Epoxy resin | | (B) Curing agent | | (C) Rubber component | | | Evaluation results | |
|---|---|---|---|---|---|---|---|---|---|
| | | Parts by | | Parts by | | Type of | Parts by | Initial | Post-sterilization |
| | Type | mass | Type | mass | Type | rubber | mass | airtightness | airtightness |
| Example 26 | A-2 | 100 | B-1-7 | 44 | C-1-2 | IR | 15 | B | B |
| Example 27 | A-2 | 100 | B-1-7 | 44 | C-2-1 | IIR | 15 | A | A |
| Example 28 | A-2 | 100 | B-1-7 | 44 | C-2-2 | EPM | 15 | A | A |
| Example 29 | A-2 | 100 | B-1-7 | 44 | C-2-4 | U | 15 | S | S |
| Example 30 | A-2 | 100 | B-1-7 | 44 | C-2-5 | Si, Q | 15 | S | S |
| Example 31 | A-2 | 100 | B-1-7 | 44 | C-2-9 | FKM | 15 | A | S |
| Example 32 | A-2 | 100 | B-1-7 | 44 | C-3-1 | BR (particles) | 15 | S | S |
| Example 33 | A-2 | 100 | B-1-1 | 15 | C-1-3 | BR | 15 | B | B |
| Example 34 | A-2 | 100 | B-1-3 | 21 | C-1-3 | BR | 15 | B | B |
| Example 35 | A-2 | 100 | B-1-11 | 18 | C-1-3 | BR | 15 | B | B |
| Example 36 | A-2 | 100 | B-1-12 | 50 | C-1-3 | BR | 15 | B | B |
| Example 37 | A-2 | 100 | B-2-1 | 100 | C-1-3 | BR | 15 | B | B |
| Example 38 | A-2 | 100 | B-2-2 | 58 | C-1-3 | BR | 15 | B | B |
| Comparative Example 1 | A-2 | 100 | B-1-6 | 40 | X-1 | ACM (particles) | 15 | B | C |
| Comparative Example 2 | A-2 | 100 | B-1-12 | 50 | X-1 | ACM (particles) | 15 | C | D |
| Comparative Example 3 | A-2 | 100 | B-1-12 | 50 | None | — | — | C | D |
| Comparative Example 4 | A-2 | 100 | B-1-6 | 40 | None | — | — | C | D |
| Comparative Example 5 | A-2 | 100 | B-1-6 | 40 | X-1 | ACM (particles) | 15 | C | D |
| Comparative Example 6 | A-2 | 100 | B-2-1 | 100 | X-1 | ACM (particles) | 15 | C | D |
| Comparative Example 7 | A-2 | 100 | B-2-2 | 58 | X-1 | ACM (particles) | 15 | C | D |
| Comparative Example 8 | A-2 | 100 | B-1-6 | 40 | X-2 | ACM | 15 | C | D |

(A) Epoxy resin

A-1:

Bisphenol A diglycidyl ether (trade name: “jER 825”, manufactured by Mitsubishi Chemical Corporation, epoxy equivalent: 170)

### A-2:

Bisphenol A diglycidyl ether (trade name: “jER 828”, manufactured by Mitsubishi Chemical Corporation, epoxy equivalent: 190)

### A-3:

Bisphenol A diglycidyl ether (trade name: “jER 834”, manufactured by Mitsubishi Chemical Corporation, epoxy equivalent: 230)

### A-4:

Bisphenol F diglycidyl ether (trade name: “EPICLON 830”, manufactured by DIC Corporation, epoxy equivalent: 170)

### A-5:

Epoxy novolac resin (product number: 406775, manufactured by Sigma-Aldrich Corporation, epoxy equivalent: 170)

(B) Curing component

(1) Polyamine compounds

### B-1-1:

1,6-Hexanediamine (manufactured by Tokyo Chemical Industry Co., Ltd., active-hydrogen equivalent: 29)

(B-45, the above-described specific example of polyamine compound)

### B-1-3:

Trimethylhexamethylenediamine (manufactured by Tokyo Chemical Industry Co., Ltd., active-hydrogen equivalent: 40)

(B-46, the above-described specific example of polyamine compound)

### B-1-6:

Polyoxyalkylenediamine (trade name: D400, manufactured by Mitsui Fine Chemicals, Inc., active-hydrogen equivalent: 100)

### B-1-7:

Polyoxyalkylenetriamine (trade name: T403, manufactured by Mitsui Fine Chemicals, Inc., active-hydrogen equivalent: 73)

### B-1-11:

m-Xylylenediamine (manufactured by Tokyo Chemical Industry Co., Ltd., active-hydrogen equivalent: 34)

(B-53, the above-described specific example of polyamine compound)

### B-1-12:

HV953U (trade name, manufactured by Nagase ChemteX Corporation, polyamidoamine, active-hydrogen equivalent: 120) (2) Acid anhydride compound

### B-2-1:

4-Methylhexahydrophthalic anhydride/hexahydrophthalic anhydride =70/30 (trade name: RIKACID MH-700, manufactured by New Japan Chemical Co., Ltd.)

(AH-12 and AH-11, the above-described specific examples of acid anhydride compound)

### (3) Thiol Compound

### B-2-2:

Pentaerythritol tetrakis(3-mercaptobutyrate) (trade name: Karenz MT PE1, manufactured by SHOWA DENKO K. K.)

### (C) Rubber Component

(0) Natural rubber

C-0: Natural rubber (NR), manufactured by Kyowa Pharmaceutical Industry Co., Ltd.

### (1) Diene Synthetic Rubbers

### C-1-1:

Styrene-butadiene rubber (SBR) (trade name: JSR 1502, manufactured by JSR Corporation)

### C-1-2:

Isoprene rubber (IR) (trade name: JSR IR2200, manufactured by JSR Corporation)

### C-1-3:

Butadiene rubber (BR) (trade name: JSR BRO1, manufactured by JSR Corporation)

### C-1-4:

Chloroprene rubber (CR) (trade name: DENKA CHLOROPRENE A90, manufactured by Denka Company Limited)

### C-1-5:

Acrylonitrile-butadiene rubber (NBR) (trade name: N280, manufactured by JSR Corporation)

### (2) Non-diene Synthetic Rubbers

### C-2-1:

Butyl rubber (isobutylene-isoprene rubber (UR)) (trade name: JSR BUTYL 065, manufactured by JSR Corporation)

### C-2-2:

Ethylene-propylene rubber (EPM) (trade name: JSR EP11, manufactured by JSR Corporation)

### C-2-3:

Ethylene-propylene-diene rubber (EPDM) (trade name: JSR EP25, manufactured by JSR Corporation)

### C-2-4:

Urethane rubber (U) (mixture of TAKELAC A-515 and TAKENATE A-50 in 6:1 (both are manufactured by Mitsui Chemicals, Inc., trade names))

### C-2-5:

Silicone rubber (silicon rubber (Si, Q)) (trade name: KE-12, manufactured by Shin-Etsu Silicone)

### C-2-6:

Chlorosulfonated polyethylene (CSM) (trade name: TS-430, manufactured by Tosoh Corporation)

### C-2-7:

Chlorinated polyethylene (CM) (trade name: ELASLEN 353A, manufactured by SHOWA DENKO K. K.)

### C-2-8:

Epichlorohydrin rubber (CO, ECO) (trade name: EPICHLOMER H, manufactured by OSAKA SODA CO., LTD.)

### C-2-9:

Fluororubber (FKM) (trade name: DAI-EL G-701, manufactured by DAIKIN INDUSTRIES, LTD.)

(3) Particulate rubbers
C-3-1: Particulate rubber (trade name: METABLEN C-223A, manufactured by Mitsubishi Chemical Corporation, butadiene rubber (BR) core-shell particles, volume-average particle size: 300 nm)
C-3-2: Particulate rubber (trade name: KANE ACE MX-960, manufactured by KANEKA CORPORATION, dispersion of silicone rubber (Si, Q) core-shell particles in liquid bisphenol A epoxy resin, formulation amount of core-shell rubber particles: 25 mass %, volume-average particle size: 100 nm)

Note that the formulation amount of C-3-2 in Table is the formulation amount of the product. Thus, for the actual formulation amount of (C) rubber component relative to (A) epoxy resin, in the adhesive, 3.75 parts by mass of silicone rubber particles were added to the total of 100 parts by mass of the epoxy resin and 11.25 parts by mass of the KANE ACE epoxy resin.

### Other Comparative Compounds

X-1: Acrylic rubber (ACM) particles (trade name: AC-3365, manufactured by Aica Kogyo Company, Limited, volume-average particle size: 500 nm) X-2: Acrylic rubber (ACM) (trade name: Nipol AR31, manufactured by ZEON CORPORATION)

As described in Table above, the epoxy adhesives of Comparative Examples 3 and 4 do not contain rubber components. The cured products obtained by curing the adhesives of Comparative Examples 3 and 4 had poor airtightness even at the initial state.

The epoxy adhesives of Comparative Examples 1, 2 and 5 to 8 contain, as the rubber component, an acrylic rubber or acrylic rubber particles. Of these, the cured products obtained by curing the adhesives of Comparative Examples 2 and 5 to 8 had poor airtightness even at the initial state. The cured product obtained by curing the epoxy adhesive of Comparative Example 1 also tended to undergo degradation of the airtightness due to repeated hydrogen peroxide plasma sterilization treatments.

By contrast, the epoxy adhesives of Examples 1 to 38, which contained a rubber component according to the present invention, have demonstrated that the cured products obtained by curing the adhesives include sufficient initial airtightness and maintain sufficiently the airtightness even after repeated hydrogen peroxide plasma sterilization treatments.

Among Examples above, of the cured products obtained from the adhesives of Examples 1 to 21, which have the same conditions except for the type of the rubber component, Examples 11 to 19 containing the non-diene synthetic rubber components had high durability for repeated hydrogen peroxide plasma sterilization treatments. In addition, the cured products obtained from the adhesives of Examples 20 and 21 containing the particulate rubber components have demonstrated that sufficient airtightness was maintained even after being subjected to the hydrogen peroxide plasma sterilization treatment performed for 100 cycles.

The present invention has been described together with embodiments thereof;

however, we do not intend to limit our invention in any minor portion of the descriptions unless otherwise specified; we believe that the invention is construed broadly without departing from the spirit and scope of the invention described in the attached claims.

### REFERENCE SIGNS LIST

electronic endoscope (endoscope)

insertion section

3a flexible tube

3b angle portion

3c distal-end portion

main-body operation section

universal cord

spiral tube

11a metal strip

sleeve mesh body

metal cap

flexible-tube base

14a distal-end side

14b base-end side

resin layer

coating layer

adhesive-cured-product layer

illumination window

observation window

forceps port

nozzle

distal-end-portion main body

distal-end cap

lens holder

prism

substrate

solid image pickup element

adhesive cured product

adhesive cured product

observation unit

What is claimed is:

1. An adhesive for an endoscope, the adhesive comprising an epoxy resin including at least one of bisphenol A epoxy resin, bisphenol F epoxy resin, or phenol novolac epoxy resin, a curing component, and a rubber component,
wherein the rubber component is not acrylic rubber.

2. The adhesive for an endoscope according to claim 1, wherein the rubber component is at least one of natural rubber, diene synthetic rubber, or non-diene synthetic rubber.

3. The adhesive for an endoscope according to claim 1, wherein the rubber component is at least one of natural rubber, styrene-butadiene rubber, isoprene rubber, butadiene rubber, chloroprene rubber, acrylonitrile-butadiene rubber, butyl rubber, ethylene-propylene rubber, ethylene-propylene-diene rubber, urethane rubber, silicone rubber, fluororubber, chlorosulfonated polyethylene, chlorinated polyethylene, polysulfide rubber, or epichlorohydrin rubber.

4. The adhesive for an endoscope according to claim 1, wherein the rubber component is non-diene synthetic rubber.

5. The adhesive for an endoscope according to claim 1, wherein the rubber component is particulate.

6. The adhesive for an endoscope according to claim 1, wherein the curing component is a polyamine compound.

7. The adhesive for an endoscope according to claim 6, wherein the curing component is a polyether-polyamine compound.

8. The adhesive for an endoscope according to claim 1, being used as a sealing material.

9. A cured product provided by curing the adhesive for an endoscope according to claim 1.

10. An endoscope comprising a constituent member fixed using the cured product according to claim 9.

11. A method for producing an endoscope, the method comprising fixing a constituent member using the adhesive for an endoscope according to claim 1.
